(19) United States
(12) Patent Application Publication
(10) Pub. No.: US 2011/0184067 A1
(43) Pub. Date: Jul. 28, 2011

(54) O-DESMETHYLVENLAFAXINE SUCCINATE POLYMORPH & PROCESS FOR PREPARING THEREOF

(75) Inventors: Ashesh Kamalnayan Pandya, Ahmedabad (IN); Chirag Girishkumar Naik, Ahmedabad (IN); Sachin Panditrao Sawant, Ahmedabad (IN)

(73) Assignee: INTAS PHARMACEUTICALS LTD., Ahmedabad (IN)

(21) Appl. No.: 13/012,129

(22) Filed: Jan. 24, 2011

(30) Foreign Application Priority Data
Jan. 25, 2010 (IN) 189/MUM/2010

(51) Int. Cl. A61K 31/194; C07C 55/02; A61P 25/24

(52) U.S. Cl. 514/574; 562/590

(57) ABSTRACT

This disclosure relates to a crystalline form of O-desmethylvenlafaxine and a process for its preparation. The disclosed crystalline form of O-desmethylvenlafaxine can be used for the manufacture of pharmaceutical compositions for the treatment of depression.

### FIELD

The present disclosure relates to a novel crystalline polymorph of Desvenlafaxine succinate.

### BACKGROUND

Desvenlafaxine succinate, also known as O-desmethyl venlafaxine (ODV) succinate is a succinate salt of ODV. ODV is a major metabolite of venlafaxine, and has been shown to inhibit norepinephrine and serotonin uptake. Synthesis of venlafaxine, its derivatives and its various salts have been disclosed in several publications (U.S. Pat. No. 4,535,186, WO 00/76955, U.S. Pat. Nos. 6,197,828, 6,689,912, 6,673,838 and 7,026,508).

U.S. Pat. No. 6,673,838 discloses five polymorphs of ODV succinate (four crystalline polymorphs and one amorphous polymorph) and processes for their preparation. Out of the five disclosed forms, form I and II are crystalline monohydrate, form III is crystalline hydrate (with water content between hemi and monohydrate), form IV is crystalline anhydrous form and an amorphous form.

Several other polymorphs are reported in various prior arts such as WO2009010990 (from A & B), WO2009009665 (Form V and form VI), WO2008017886 (hydrate), WO2009118758 (Form V, VI, VII), WO2009010990, US200818856 (Form F), and WO2008110338 (Form V).

New crystalline polymorph of a drug substance may have different physical and chemical properties such as melting point, hygroscopicity, stability, solubility and/or dissolution rate, crystallinity, crystal habits, bioavailability, chemical reactivity and formulation handling characteristics, which are among the numerous properties that need to be considered in preparing medicament that can be effectively administered. Therefore, the regulatory agencies require a definitive control of polymorphic form of the active component in solid pharmaceutical dosage forms.

Accordingly, there is an ongoing need to search new polymorphic forms of ODV succinate that have good thermal stability and material flow character, lower water contents, and offer advantages for preparing reproducible pharmaceutical formulations. The disclosed polymorphic form of ODV succinate helps fulfill this and other needs.

### SUMMARY

The main object of the present disclosure is to provide a novel crystalline polymorphic form of O-desmethylvenlafaxine succinate.

Another object of the present disclosure is to provide a process for preparing a novel crystalline polymorphic form of O-desmethylvenlafaxine succinate.

The present disclosure describes a crystalline form Z of O-desmethylvenlafaxine succinate, which is characterized by an X-ray diffraction pattern comprising 2 theta values at 20.02, 26.12 and 31.45±0.2 degrees, wherein the peak at 2 theta value 31.45±0.2 degrees is at medium relative intensity. The disclosed crystalline polymorph of O-desmethylvenlafaxine succinate is further characterized by an X-ray diffraction pattern with 2 theta values at 13.27, 15.95 and 20.51±0.2 degrees.

The present disclosure describes a process for preparing crystalline form Z comprising:
(a) forming a suspension of O-desmethylvenlafaxine and succinic acid, in a mixture of methylene dichloride, water and hexane or cyclohexane
(b) heating the suspension;
(c) cooling the suspension of step b to obtain crystalline form Z of O-desmethylvenlafaxine.

### BRIEF DESCRIPTION OF THE DRAWING

FIG. 1 shows X-ray diffraction pattern corresponding to form Z of O-desmethylvenlafaxine.

### DETAILED DESCRIPTION

The disclosed crystalline form Z of O-desmethylvenlafaxine succinate is characterized by an X-ray diffraction pattern comprising 2 theta values at 20.02, 26.12 and 31.45±0.2 degrees; wherein the peak at 2 theta value 31.45±0.2 degrees is at medium relative intensity.

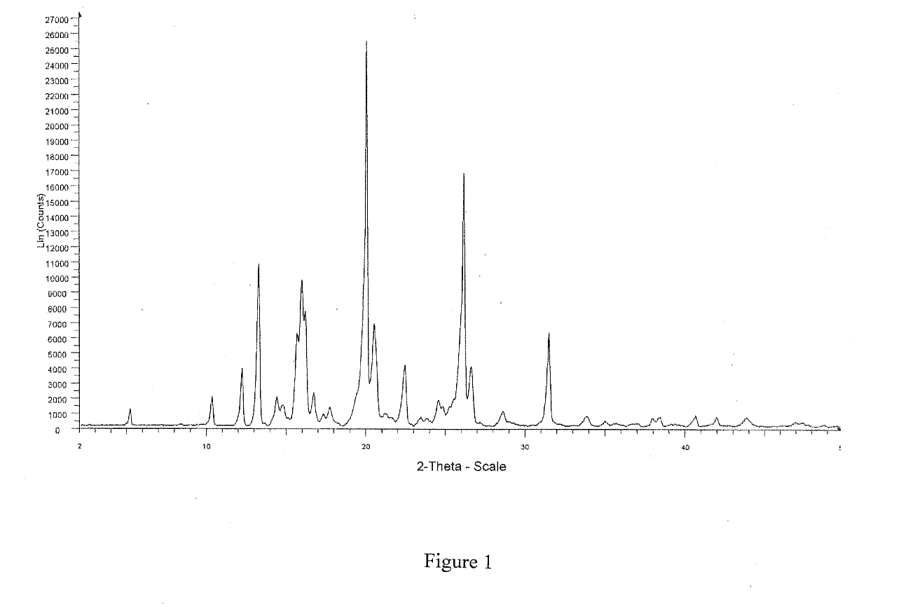

In one embodiment the crystalline form Z of O-desmethylvenlafaxine succinate has an X-ray diffraction pattern substantially identical to that shown in FIG. 1. Peak locations and intensities for the X-ray diffraction pattern in FIG. 1 are provided in table 1 below.

TABLE 1

| Degrees 2θ | Relative intensity |
|---|---|
| (±2θ) | % |
| 12.15 | 13.9 |
| 13.27 | 29.2 |
| 15.95 | 55.4 |
| 20.02 | 100 |
| 20.45 | 47.8 |
| 26.12 | 36.8 |
| 31.45 | 30.7 |

The disclosed crystalline polymorph of O-desmethylvenlafaxine succinate is further characterized by an X-ray diffraction pattern with 2 theta values at 13.27, 15.95 and 20.51±0.2 degrees. The water content of the form Z can be between 0 to 0.25 mole %.

In another embodiment the crystalline form Z of O-desmethylvenlafaxine succinate has water content of about 0.05%-0.25 mole %. The crystalline form Z of O-desmethylvenlafaxine is a stable form and retains its X-ray diffraction characteristics for at least six months.

According to another aspect, the disclosed crystalline polymorph is prepared in a non-polar solvent system comprising of a mixture of methylene dichloride (MDC)-Hexane-water or methylene dichloride-Cyclohexane-water.

In an embodiment the process to prepare form Z of the present application comprises:
(a) forming a suspension of O-desmethyl venlafaxine and succinic acid in a mixture of methylene dichloride, water and hexane or cyclohexane;
(b) heating the suspension to about 40° C.-80° C.;
(c) cooling the suspension to about 0° C.-35° C.; and
(d) filtering the suspension to isolate the O-desmethyl venlafaxine form Z.

The suspension obtained in step a) is heated at or below the reflux temperature of solvent mixture. Preferably the suspension or reaction mixture is heated at a temperature in range of about 40° C.-80 C. More preferably the reaction mixture is heated in the range of about 35° C.-45° C. The time of heating may vary from 15 minutes to about 5 hours.

Preferably the reaction mixture is cooled to isolate form Z of O-desmethylvenlafaxine. The reaction mixture may be cooled to ambient temperature to about 0° C., and can be maintained at the same till complete separation of the crystals.

The crystals thus obtained may be separated from the solution by filtration, decantation, centrifugation or any other method known in the art. The crystals thus obtained can be dried at atmospheric pressure or under vacuum.

In one embodiment of a pharmaceutical composition, the composition comprises the crystalline form Z described above and at least one pharmaceutically acceptable excipient. In another embodiment, the pharmaceutical composition is useful for the treatment of depression.

The disclosed crystalline form Z of O-desmethylvenlafaxine succinate can be formulated into the conventional dosage forms for peroral or parenteral administration. Tablets and capsules are preferred formulation. They can be produced by conventional mixing processes and with the use of conventional pharmaceutical excipients. Pharmaceutically acceptable excipients comprises of binders, disintegrants, flavoring agents and the likes thereof.

Relative intensity is defined as the ratio of the peak intensity to that of the most intense peak. Medium relative intensity is defined as the ratio of the peak intensity to that of the most intense peak, wherein the calculated ratio is in the range of about 10 to about 35%.

### EXAMPLES

### Example 1

15.0 g O-desmethyl venlafaxine free base and 6.72 g succinic acid were charged in to the mixture of 45 ml methylene dichloride and 135 ml hexane under stirring. 0.2 ml water was added in to the reaction mixture. The temperature of reaction mixture was raised to 42-45° C. and maintained at the same for 3.0 hours. After that, heating was stopped and reaction mixture was allowed to cool to ambient temperature, filtered the solid and washed with 30 ml of hexane. The solid was dried at 50-55° C. under vacuum.

Yield—21.5 g; Assay—99.5%

### Example 2

500 g O-desmethylvenlafaxine free base and 224.18 g succinic acid were charged in to the mixture of 1500 ml dichloromethane and 4500 ml cyclohexane under stirring. 6.5 ml water was added in to the reaction mixture. The reaction mixture was heated at 42-45° C. for 3.0 hours. After that, heating was stopped and reaction mixture was allowed to cool to ambient temperature, filtered the solid and washed with 1000 ml of cyclohexane. The solid was dried at 50-55° C. under vacuum.

Yield—950 g; Assay—100.7%

X-Ray Powder Diffraction (XRPD)

### Instrument and Settings

### Instrument: D-8 Advance

### Make: Bruker AXS

### Detector: Lynx eye

### X-ray Parameter:

X-ray tube: Copper KαA
Kβ filter: Ni

### Wavelength: 1.5406 Å°

### Scan Parameter:

Scan type: Locked coupled
Scan mode: Continuous
Scan axis start 2θ: 20
Scan axis stop 2θ: 500
Scan speed: 0.5 sec
Scan step size: 0.03°

### Motorized Slits:

Divergence slit: 0.3°
Ant scattering slit: Fixed
Rotation: On (30 rpm)

### Generator Parameter:

### Voltage: 40 kV

### Current: 30 mA

### Method

Place the substance being examined on the sample holder; pack and smooth its surface with a polished glass microscope slide and record the diffractogram. Compare the XRPD pattern of sample with XRPD pattern of Desvenlafaxine working standard, similarly determined.

We claim:

1. A compound which is a crystalline form Z of O-desmethylvenlafaxine succinate, wherein the compound exhibits an X-Ray powder diffraction pattern, expressed in degree 2θ (±0.2° 2θ), having characteristic peak of medium relative intensity at 31.45 (±0.2° 2θ).

2. Compound of claim 1, wherein the crystalline form further exhibits an X-Ray powder diffraction pattern, expressed in degree 2θ (±0.2° 2θ), having characteristic peaks at 20.02 and 26.12.

3. Compound of claim 1, wherein the crystalline form further exhibits an X-Ray powder diffraction pattern, expressed in degree 2θ (±0.2° 2θ), having characteristic peaks at 13.27, 15.95 and 20.51.

4. Compound of claim 1, wherein the medium relative intensity is in the range of about 10 to about 35%.

5. A process for preparing crystalline form Z of O-desmethylvenlafaxine succinate comprising:
a. forming a suspension of O-desmethyl venlafaxine, succinic acid in a mixture of methylene dichloride, water and hexane or cyclohexane;
b. heating the suspension to a temperature of about 40° C. to about 80° C.;
c. cooling the suspension to a temperature of about 0° C. to about 35° C.

6. A pharmaceutical composition, comprising:
the compound of claim 1; and
a pharmaceutically acceptable carrier or excipient.

7. The pharmaceutical composition of claim 6, wherein the pharmaceutical composition comprises an amount of the compound of claim 1 effective for treating depression in a subject in need thereof.
